(19) United States
(12) Patent Application Publication
(10) Pub. No.: US 2006/0200183 A1
(43) Pub. Date: Sep. 7, 2006

(54) Multi-functional medical instrument and methods of use

(76) Inventor: Raymond J. Gardocki, Germantown, TN (US)

Correspondence Address: BROWN RAYSMAN MILLSTEIN FELDER & STEINER, LLP, 1880 CENTURY PARK EAST, 12TH FLOOR, LOS ANGELES, CA, 90067, US

(21) Appl. No.: 11/070,110

(22) Filed: Mar. 1, 2005

(51) Int. Cl. A61B 17/00

(52) U.S. Cl. 606/190

(57) ABSTRACT

Disclosed herein are medical instruments having an elongate member having a first end, a second end, and a bore extending the length of the elongate member, a body is coupled to the second end of the elongate member where the body having a suctioning control means in communication with the bore of the elongate member, and a fenestrated tip coupled to the first end of the elongate member, wherein the fenestrated tip is sufficiently rigid to dissect and retract tissues and prevents obstruction of the bore.

### BACKGROUND

Various medical instruments have been designed and developed for use in surgical procedures. Typically, these instruments are specialized for particular uses during surgical procedures. For instance, a suctioning device is used to remove fluids such as blood from solid debris such as tissue, bone fragments, or the like. Other specialized devices such as forceps, dissectors, probes, and the like are used to move, separate, and retract tissues.

While these specialized devices are useful for performing particular functions, requiring and switching between a plurality of instruments can be a time consuming process for a surgeon. Additionally, there may be situations where the surgeon would not have the use of a free hand and the assistance of other medical staff may not be practical or possible. Thus, the use of many instruments during a surgical procedure can increase the duration of the surgical procedure, which subjects the patient to longer periods of anesthetization. Accordingly, multi-functional instruments have been developed in art. However, there still remains a need for multi-functional instruments that reduces the number of instruments required by a surgeon during a medical procedure.

### SUMMARY

Briefly, and in general terms, various embodiments of a multi-functional instrument are disclosed herein. The multi-functional instrument includes a padded and porous tip, an elongate body, and a suction control means. The multi-functional instrument is a handheld instrument that can be used to dissect or retract tissue, and remove liquids from solid debris from the surgical field without becoming obstructed. The instrument may be used in open, minimally invasive, or laparoscopic procedures. Additionally, the instrument may be a single use device or a reusable device. In use, a surgeon can perform these functions with one hand thereby allowing free use of the surgeon's other hand while not having to pause to unclog the instrument.

### BRIEF DESCRIPTION OF THE DRAWINGS

FIG. 1 is a top plan view of one embodiment of the multi-functional medical instrument;

FIG. 2 is a side view of an alternate embodiment;

FIG. 2A is a fragmented top plan view of one embodiment of a suction control means for a multi-functional medical instrument;

FIG. 2B is a fragmented top plan view of another embodiment of a suction control means for a multi-functional medical instrument;

FIG. 3 is a side view of another embodiment of the multi-functional medical instrument;

FIG. 4 is a cross-sectional view of multi-functional medical instrument of FIG. 3 taken along line 4-4;

FIG. 5 is a cross-sectional view of multi-functional medical instrument of FIG. 3 taken along line 5-5;

FIG. 6 is a fragmented side view of an embodiment of a multi-lumen tube for a multi-functional medical instrument;

FIG. 7 is a fragmented side view of another embodiment of a multi-lumen tube for a multi-functional medical instrument;

FIG. 8 is top plan view of another embodiment of the multi-functional medical instrument;

FIG. 9 is a side view of embodiment depicted in FIG. 8;

FIG. 10 is a fragmented side view of an embodiment of a suction controller for a multi-functional medical instrument;

FIG. 11 is a fragmented side view of another embodiment of a suction controller for a multi-functional medical instrument; and

FIG. 12 is a fragmented side view of yet another embodiment of a suction controller for a multi-functional medical instrument.

### DESCRIPTION OF EMBODIMENTS

The embodiments disclosed herein are directed to a multi-functional medical instrument that may be used in open, minimally invasive, or laparoscopic procedures. The multi-functional instrument is a handheld instrument that can be used to dissect or retract tissue, and remove liquids from solid debris from the surgical field without becoming obstructed. In use, a surgeon can perform these functions with one hand thereby allowing free use of the surgeon's other hand while not pausing to unclog the instrument.

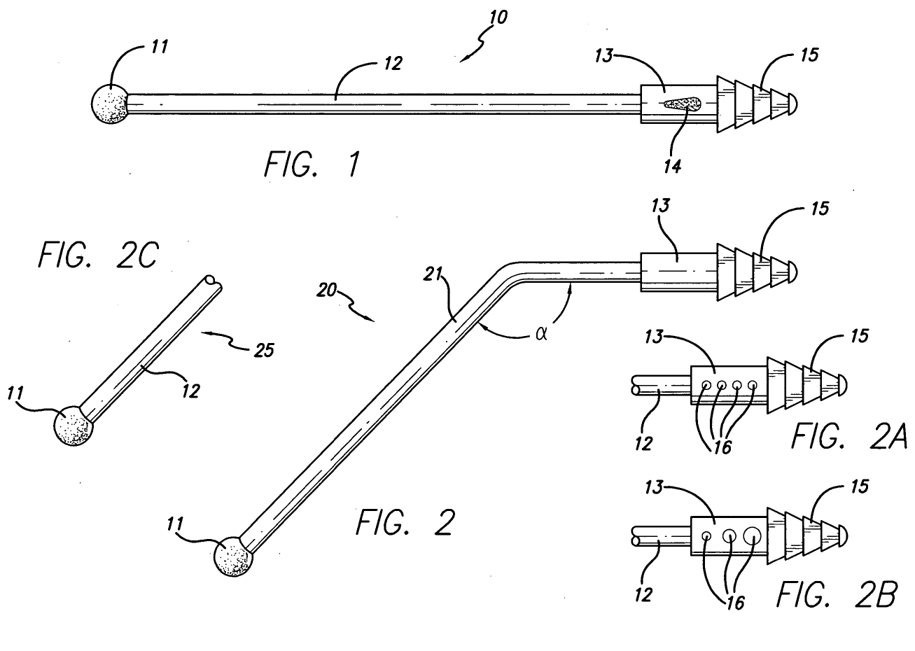

FIG. 1 depicts one embodiment of the multi-functional medical instrument 10. Generally, the multi-functional medical instrument 10 includes a tip 11, a body 12, a suctioning means 14, and a connector 15. The instrument 10 may be coupled to a vacuum source (not shown) via the connector 15. Of course, one of ordinary skill in the art will appreciate that not all multi-functional medical instruments 10 will have all these components, and may, indeed, have other components in addition to or in lieu of those components mentioned here.

The tip 11 is made of a fenestrated, perforated, or woven material, which allows the suction of liquids and not solids and prevents the obstruction of the suctioning channel. The tip material also provides padding to the end of the instrument 10, which prevents damage to structures when pressure is applied. The material that makes up the tip 11 has sufficient density to allow the tip to be used as a dissector and/or retractor. That is, the tip material has sufficient rigidity so that the tip 11 does not collapse when pressure is applied to the tip. The tip 11 has a coefficient of friction which eases separation of tissues. Accordingly, the tip 11 can be used for tissue dissection or to retract delicate structures such as, but not limited to, nerve tissue, vascular tissue, bowel, or the gall bladder. As shown in FIG. 1, the tip 11 has a generally rounded shape. As those skilled in the art will appreciate, the tip may be configured in any shape known or developed in the art.

As shown in FIG. 1, the tip 11 is coupled to a tube 12 by a coupling means. In one embodiment, the tip 11 may be coupled to the tube 12 by an adhesive. In another embodiment, the tip 11 may be coupled to the tube 12 by a friction fit. In yet another embodiment, the tip 11 may be molded directly to the tube 12. As those skilled in the art will appreciate, the tip 11 may be coupled to the tube by any means known or developed in the art. In these embodiments, the tip 11 is permanently coupled to the tube 12. Give the porous nature of the tip 11, these instruments 10 cannot be cleaned for reuse and are generally single use instruments.

By reversibly coupling the tip 11 to the end of the tube 12, the tip 11 may be removed from the end of the tube 12 so that that the remaining portion of the multi-functional device 10 may be reused and a new tip 11 may then be applied to the end of the tube. According to one embodiment, the tip 11 may be reversibly coupled to the end of the tube 12. For instance, the tip 11 may be sewn onto the end of the tube 12 with nylon thread or other thread known or developed in the art. In another embodiments, the tip 11 may be coupled to the tube 12 by nylon ties or the like. In this embodiment, the proximal end of the tip 11 may include loops or slits that are sized to receive the nylon ties. In other embodiments, the tip 11 may be attached to the tube 12 via a friction fit or detachable coupler.

In FIG. 1, the tube 12 is generally an elongate body having a bore. As those skilled in the art will appreciate, the tube 12 can have varying lengths, diameters, and one or more lumens. As shown in FIG. 1, the tube 12 has a uniform diameter. In other embodiments, the tube 12 may be tapered. In another embodiment, the tube 12 may be only tapered at the end of the tube by the tip 11. FIG. 2 depicts another embodiment of a multi-functional instrument 20 having a tube 21 bent at a fixed angle α. It is contemplated that the angle a may be between approximately 0° C. to approximately 90° C. As those skilled in the art will appreciate, the tube 12 can be made from a plurality of materials such as, but not limited to, polycarbonate, polypropylene, or other plastics, fiberglass or stainless steel.

In FIG. 1, the tube 12 is coupled to a suction control means 13. As shown in FIG. 1, the suction control means 13 is a hollow plenum having an opening 14 and is coupled to a connector 15. The suction control means 13 may be a box-like structure having a rectangular, cubic, or any other polygonal shape having at least one flat surface. Generally, the suction control means 13 has a greater cross-section than the tube 12 in order to provide a collection area for clotted fluids thereby minimizing clotting of the instrument 10. Additionally, the larger size of the suction control means 13 provides a larger surface area for the user to grasp for user comfort. However, in one embodiment, the suction control means 13 may have the same cross-sectional area as the tube 12.

In FIG. 1, the connector 15 coupled to the end of the suction control means 13 and is sized to accommodate various sizes of tubing that is in communication with the vacuum source (not shown). Generally, the connector 15 is integral with the suction control means 13, but it is contemplated that the connector 15 may be a separate component that is coupled to the suction control means. As shown in FIGS. 1-2, the connector 15 is tapered and includes barbs to securely grasp the tubing. In other embodiments, the connector may have a uniform outside diameter and may or may not include barbs as shown in FIG. 1. Accordingly, in these embodiments, nylon ties, threads or detachable couplers (none shown) may be used to secure the tubing to the connector.

The suction control means 13 also includes an opening 14 that controls the suction force at the tip 11. As shown in FIG. 1, the opening 14 is tear-shaped. In other embodiments, it is contemplated that the opening 14 may have any shape known or developed in the art. In another embodiment, the suction control means 13 may include one or more openings 16 that are generally aligned along a common line. For instance, as shown in FIG. 2A, the openings 16 may have similar diameters or have varying diameters as depicted in FIG. 2B. Accordingly, in use, maximum suctioning force is obtained when the user covers all the openings and as each opening is exposed to ambient environment, the suctioning force is reduced.

In other embodiments, the suction control means 13 may be a spring-loaded trumpet valve 100, a rotating thumb wheel 110, or a sliding lever 120 as shown in FIGS. 10-12. As those skilled in the art will appreciate, the suction control means 13 can be any means to control the suction force at the tip 11 of the multi-functional instrument that has been known or developed in the art.

In use, the suction forces at the tip 11 of the instrument is controlled by varying the amount of the opening 14 that is covered by the user's finger. If the opening 14 is completely covered, then maximum suctioning force is available at the tip of the instrument. Alternatively, if a portion of the opening 14 is exposed to the ambient environment, the suctioning force available at the tip of the instrument is reduced.

It is also contemplated that one embodiment of the multi-functional device 25 is composed of the tip 11 coupled to the tube 12 as shown in FIG. 2C. The tube 12 has a diameter slightly larger than the outside of the diameter of a standard suctioning device (not shown). In use, the tube 12 of the device 25 may be friction fitted over the existing suction device. Accordingly, an existing suctioning device retrofitted with the multi-functional device 25 may be used to dissect or retract tissue while removing fluids from the surgical field.

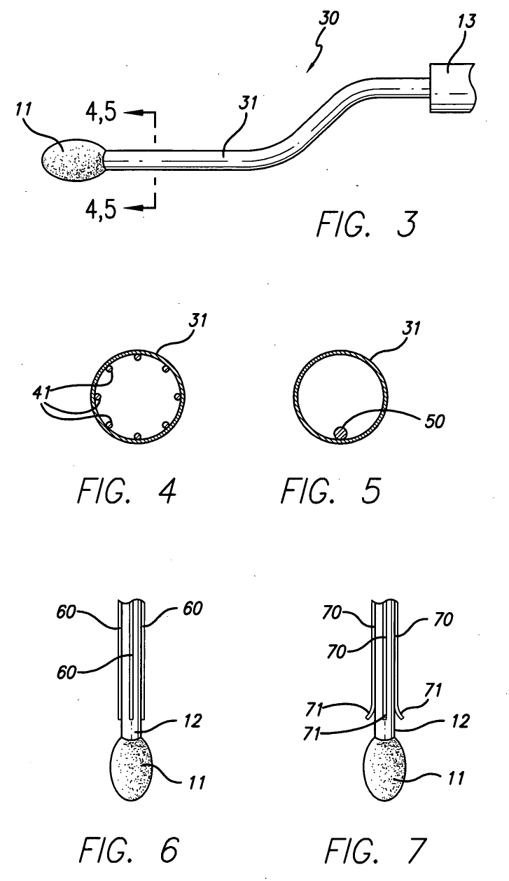

Turning now to FIG. 3, yet another embodiment of a multi-functional instrument 30 is illustrated. The instrument 30 has a flexible tube 31 that may be bent to any angle or configuration. For instance, as shown in FIG. 3, the tube 31 is bent in a S-shaped configuration. In another configuration, the tube 31 may be bent in a J-shaped configuration. FIGS. 4 and 5 illustrate two cross-sectional views of the tube 31. In FIG. 4, the tube 31 is reinforced with a malleable wire 41 that allows the tubing 31 to be adjustable yet capable of holding various shapes while maintaining the patency of the lumen(s). In FIG. 5, the tube 31 includes a single malleable wire 40 that may be used to maintain the configured shape of the tube 31. In yet another embodiment, one or more malleable wires may be embedded within the walls of the tube. In another embodiment, one or more malleable wires may be positioned on the outside of the tube. With respect to the embodiment disclosed above, it is contemplated that the malleable wires may extend the entire length of the tube. In alternate configurations, the malleable wires may be located on one or more sections of the tube. For instance, in one embodiment, the malleable wires may be positioned near the tip of the device.

As shown in FIGS. 1-5, the instrument 10 has a tube 12 having a single lumen. In other embodiments, the tube 12 may have more than one lumen. For instance, the tube 12 may have walls that extend the length of the lumen to bifurcate the tube into multiple channels that provide suctioning, irrigation, and medicament delivery. FIG. 6 illustrates another embodiment of the multi-functional medical instrument having a plurality of secondary tubes 60 coupled to the external surface of the tube 12. Generally, the secondary tubes 60 extend the length of the instrument and terminate near the tip 11. The secondary tubes 60 are generally smaller in diameter than the main tube 12. However, it is contemplated that the diameter of secondary tubes 60 may be varied. As shown in FIG. 6, the secondary tubes 60 are spaced about the circumference of the tube 12. FIG. 7 illustrates yet another embodiment where the ends 71 of the secondary tubes 70 are flared away from the surface of the tube 12. As those skilled in the art will appreciate, the secondary tubes may be varied in number, position, point of termination, or diameter from what is depicted in FIGS. 6-7 depending on the purpose of the secondary tubes.

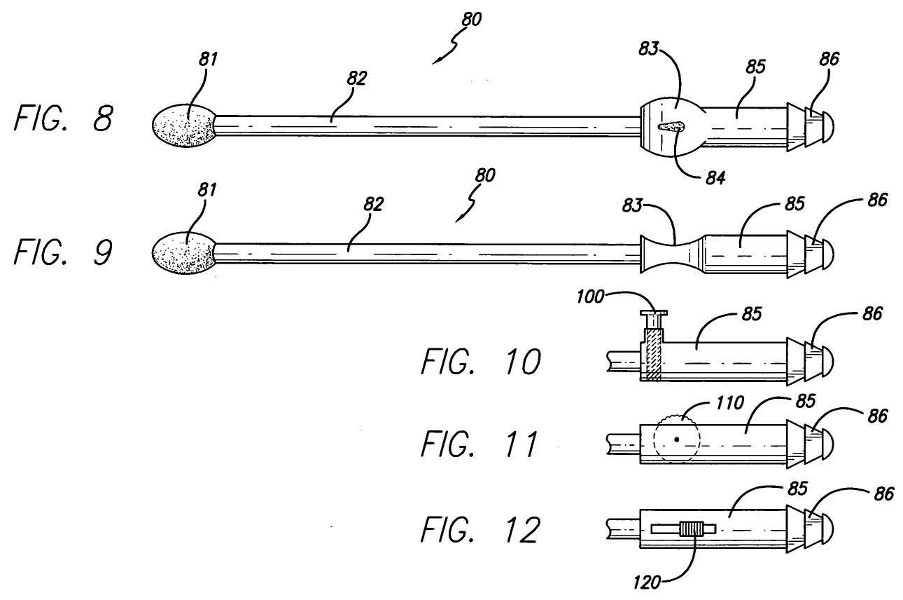

FIGS. 8 and 9 illustrate another embodiment of the multi-functional medical instrument 80. These figures show an instrument 80 that is intended for use in laparoscopic procedures. That is, this instrument 80 is held like a pencil or knife rather than a drumstick. Like the previous embodiments shown in FIGS. 1 and 2, the instrument 80 shown in FIGS. 8 and 9 include a tip 81, tube 82, and suction control means 84. The instrument 80 includes an enlarged area 83 around the suction control means 84 as shown in FIG. 8. The enlarged area may be curved as shown in FIG. 9, but other embodiments where the enlarged area 83 is flat. Furthermore, the side opposite the enlarged area 83 may also be curved. The curved surfaces allow the user to comfortably hold the instrument 80. The instrument 80 also includes an accumulator 85 having a generally cylindrical structure that is coupled to the suction control means. However, as those skilled in the art will appreciate, the accumulator may have any shape known or developed in the art. As shown in FIGS. 8-9, the accumulator 85 also has a diameter larger than the tube 82. The accumulator 85 is a collection area for clotted fluids thereby minimizing clotting of the instrument 10. Furthermore, the larger diameter also allows the user to comfortably hold the instrument 80. In an alternate embodiment, the accumulator 85 may have the same diameter as the tube 80. In yet another embodiment, the instrument 80 may be configured so that it does not include an accumulator.

As those skilled in the art will appreciate, the various embodiments of the components of the multi-functional instrument depicted in the figures may be combined or substituted to form other multi-functional instruments that are not depicted or explicitly illustrated or disclosed herein.

In use, a vacuum source is placed in communication to the multi-functional medical instrument 10 via tubing (not shown), which, in turn is coupled to the connector 15. In particular embodiments, nylon ties or other coupling means such as threads or friction may be used to ensure that the tubing is securely coupled to the connector. Accordingly, the vacuum source creates a suction force at the tip 11 of the instrument 10. The suctioning force at the tip 11 of the instrument 10 may be adjusted with the suction control means 13. In some embodiments, the suctioning force is controlled by adjusting the amount or number of openings 14 that are exposed to the ambient environment. Whether or not a suctioning force is present, the surgeon may use the tip 11 to dissect or retract soft tissue or other delicate structures. However, it is possible that the surgeon may use the suctioning force to retract or hold a structure. Any fluid in the surgical field is then drawn into the instrument 10 through the tip 11. The tip 11 acts as a filter preventing any loose debris from being drawn into the instrument 10. Any fluid that clots or coagulates in the instrument may be collected in the accumulator 13. The fluid may then be accumulated in a reservoir or similar structure downstream of the instrument 10.

In closing, it is to be understood that the embodiments disclosed herein are illustrative and other modifications that may be employed are within the scope of the specification. Thus, by way of example, but not of limitation, alternative configurations may be utilized in accordance with the teachings herein. Accordingly, the drawings and the description are illustrative and not intended to be a limitation thereof.

What is claimed:

1. A medical instrument, comprising:
an elongate member having a first end, a second end, and a bore extending the length of the elongate member;
a body coupled to the second end of the elongate member, the body having a suctioning control means in communication with the bore of the elongate member; and
a fenestrated tip coupled to the first end of the elongate member, wherein the fenestrated tip is sufficiently rigid to dissect and retract tissues and sufficiently soft to minimize damage to delicate structures.

2. The medical instrument of claim 1, further comprising a connector coupled to the body.

3. The medical instrument of claim 1, further comprising one or more tubes coupled to the elongate member.

4. The medical instrument of claim 3, wherein the ends of one or more tubes are flared away from the elongate member.

5. The medical instrument of claim 1, wherein a portion of the elongate body is bent at an angle between approximately 0° to approximately 90°.

6. The medical instrument of claim 1, wherein a portion of the elongate body is flexible.

7. The medical instrument of claim 1, wherein a portion of the elongate body includes one or more malleable wires.

8. The medical device of claim 1, wherein the suction control means is one or more openings, a trumpet valve, a rotating thumb wheel, or a sliding lever.

9. The medical device of claim 8, wherein the one or more openings have approximately the same diameter.

10. The medical device of claim 8, wherein the one or more openings have differing diameters.

11. A medical device, comprising:
a means for controlling a suction force; and
a means for dissecting and retracting tissue, wherein the suction controlling means and the dissecting and retracting means are operable with one hand of a user.

12. The medical device of claim 11, wherein the controlling means is one or more openings, a trumpet valve, a rotating thumb wheel, or a sliding lever.

13. The medical device of claim 12, wherein the one or more openings have approximately the same diameter.

14. The medical device of claim 12, wherein the one or more openings having differing diameters.

15. The medical device of claim 11, wherein the dissecting and retracting means is a fenestrated tip coupled to an elongate member having a bore, wherein the fenetrated tip is adapted to be a filter.

16. The medical device of claim 15, wherein the dissecting and retracting means prevents obstruction of the bore providing the suction force.

17. The medical device of claim 15, wherein the dissecting and retracting means has a coefficient of friction which eases separation of tissue.

18. The medical device of claim 15, wherein the elongate member is flexible.

19. The medical device of claim 15, further comprising one or more tubes coupled to the elongate member.

20. A method for using a medical device with one hand, comprising:
providing a device having a tip with a fenestrated material;
placing the tip of the device within an area having fluids;
applying a suctioning force to the tip to remove fluids from the area;
applying a force to the tip to dissect tissues; and
retracting a structure with the tip.

21. The method of claim 20, further comprising applying a suctioning force to retract the structure.
